(19) United States
(12) Patent Application Publication
(10) Pub. No.: US 2021/0298649 A1
(43) Pub. Date: Sep. 30, 2021

(54) EARLY DETECTION OF COVID-19 IN HUMANS AND ANIMALS AND AN IMMUNOTHERAPY AGAINST VIRUSES

(71) Applicant: Kiffik Inc., Chateauguay (CA)

(72) Inventors: Abdeltif ESSALIK, St-Romuald (CA); André DUSSAULT, Quebec (CA); Samia BENSALEM, Solothurn (CH)

(21) Appl. No.: 16/835,570

(22) Filed: Mar. 31, 2020

(51) Int. Cl. A61B 5/145; A61B 5/1477; A61B 5/00

(52) U.S. Cl. CPC A61B 5/14514; A61B 5/1477; A61B 2562/0247; A61B 5/6837; A61B 5/6832

(57) ABSTRACT

It is provided a device for non-invasively and continuously extracting interstitial fluid comprising or not comprising an immunogenic compound from the skin of a subject comprising an electroporator generating a non-pulsed voltage coupled to a pulsed voltage between at least one moving electrode and an electrical conducting adhesive for non-heating irreversibly electroporating the skin; a vacuum pump providing a negative pressure above the skin; and a collecting chamber for collecting the interstitial fluid extracted from the skin wherein the device can be used for detecting Covid-19 and/or extracting an antibody produced in the subject.

### TECHNICAL FIELD

It is provided a device for non-invasively and continuously extracting interstitial fluid which may or may not comprise an immunogenic compound through the skin of a subject.

### BACKGROUND

In the 21^{st} century, a typical seasonal flu infects between 5 to 15% (340 million to 1 billion) of the world population leading to 290,000-650,000 deaths/year worldwide. The 2019-20 seasonal flu in its first quarter has already infected 10% of the world population (800 million; USA, 32 million) and caused 450,000 deaths worldwide (USA 18,000).

On the other hand, the apparition of the Covid-19, a new coronavirus originated from China, that has spread throughout the world has shadowed the typical seasonal flu season in terms of the economic burden, medical cost, and severity of the illness.

Currently, real-time polymerase chain reaction (PCR) is one of the early PCR tests for the detection of Covid-19 developed at Charité in Berlin in January 2020 using real-time polymerase chain reaction (RT-PCR) and formed the basis of 250,000 kits that the WHO is distributing.

A South Korean company, Kogenebiotech, developed a clinical grade, PCR-based 2019-nCoV detection kit called PowerChek Coronavirus. It looks for the “E” gene shared by all beta coronaviruses, and the RdRp gene specific to SARS-CoV-2. Other companies like Seegene and Solgent also developed their versions of clinical grade detection kits called DiaPlexQ™ and Allplex™ 2019-nCoV Assay respectively.

In the United States, the US Center for Disease Control is distributing the CDC 2019-Novel Coronavirus (2019-nCoV) Real-Time RT-PCR Diagnostic Panel to public health labs through its International Reagent Resource. One of three genetic tests in older versions of the test kits caused inconclusive results, and a bottleneck of testing at the CDC in Atlanta; tests using two components were determined to be reliable, allowing state and local laboratories to complete testing quickly. The test was approved by the Food and Drug Administration under an Emergency Use Authorization.

COVID-19 testing can also be done with antibody test kits. Antibody assays use blood serum sample and can provide a positive result even if the person has recovered and the virus is no longer present. The first antibody test was demonstrated by a team at the Wuhan Institute of Virology. Afterwards a team from Duke-NUS Medical School in Singapore announced another antibody test for COVID-19 that can provide a result within a few days. Subsequently, another South Korean company called PCL filed a Fast Track Designation Request to Ministry of Food and Drug Safety of South Korea for their antibody-based detection kit, COVID-19 Ag GICA Rapid. Unlike a real-time RT-PCR based detection kit, PCL claims that their antibody-based kit could make a diagnosis within 10 min.

The Explify Respiratory test by IDbyDNA is a metagenomic nucleic acid analysis test that can identify over 900 respiratory pathogens, including the SARS-CoV-2 virus strain. The actual data analysis takes less than an hour; the turnaround time from receipt of sample to test result is 36 hours. It is more expensive than PCR testing.

Chest CT scan, a routine imaging tool for pneumonia diagnosis, is fast and relatively easy to perform. One research found that the sensitivity of CT for COVID-19 infection was 98% compared to RT-PCR sensitivity of 71%. For this study, researchers at Tongji Hospital in Wuhan, China, set out to investigate the diagnostic value and consistency of chest CT imaging in comparison to RT-PCR assay in COVID-19.

Since none of the above methods could detect the presence or the absence of the Covid-19 continuously and quickly, it is thus highly desired to be provided with an efficient, fast, inexpensive and portable detection device that can detect Covid-19.

### SUMMARY

It is provided a device for non-invasively and continuously extracting interstitial fluid which may or may not comprise an immunogenic compound through the skin of a subject comprising an electroporator generating a non-pulsed voltage coupled to a pulsed voltage between at least one moving electrode and an electrical conducting adhesive for non-heating irreversibly electroporating the skin; a vacuum pump providing a negative pressure above the skin, the vacuum pump in operation providing the negative pressure with electroporating the skin to form openings in the skin for extracting the interstitial fluid, the interstitial fluid being extractable only so long as the negative pressure is applied; and a collecting chamber for collecting the interstitial fluid extracted from the skin.

In an embodiment, the device described herein comprises an impedimetric sensor for detecting the immunogenic compound.

In an embodiment, the impedimetric sensor comprises at least one of an anti-Spike antibody, anti-envelope antibody, anti-interleukin-6 antibody, a serum albumin and a promotor of adhesion.

In an embodiment, the collecting chamber comprises an extraction chamber for collecting the native interstitial fluid extracted through the skin.

In an embodiment, the device described herein comprises a sampling chamber connected to the extraction chamber by a pumping conduit, the sampling chamber collects the extracted interstitial fluid through the skin for its analysis.

In another embodiment, the device described herein comprises a pressure sensor for monitoring the pressure applied above the electroporated skin.

In another embodiment, the device described herein comprises a microcontroller connected to the pressure sensor in order to monitor and control the pressure above the skin and inside the extraction chamber.

In an embodiment, the impedimetric sensor detects the immunogenic compound by using a micro-potentiostat which apply a single frequency sinusoidal potential perturbation superimposed on a DC potential and applied to the impedimetric sensor maintained at a DC voltage.

In Another Embodiment, the Amplitude of the Sinusoidal Perturbation is 10 mV±2 mV and the Single Frequency is Comprised Between 10 mHz to 10 Hz

It is further provided a method of extracting an immunogenic compound through the skin from a subject comprising the steps of contacting the device as described herein on the skin of the subject; electroporating the skin non-invasively and continuously extracting interstitial fluid from a subject; applying a negative pressure at or near the region of the skin being electroporated, the simultaneous combination of electroporating the skin and applying the negative pressure forming openings in the skin, the interstitial fluid being extractable only so long as the negative pressure is applied; and extracting an immunogenic compound present in the interstitial fluid.

In a further embodiment, the immunogenic compound is a pathogen, such as a virus for example.

In a further embodiment, the antibody is an anti-Spike antibody, an anti-envelope antibody or Interleukin-6 antibody.

In an embodiment, the skin is electroporated by applying a voltage is between 5 V to 500 V.

In a further embodiment, the amplitude of the voltage is between 50 μS to 7000 μS.

In a particular embodiment, the subject is an animal or a human.

In accordance with the present disclosure, there is now provided a method for non-invasively and continuously extracting the native interstitial fluid from an animal or a human comprising the steps of a non-heating irreversible electroporation of the skin; applying a negative pressure above the region of the electroporated skin for extracting the native interstitial fluid; and collecting the extracted native interstitial fluid through the skin.

It is also provided a device for non-invasively and continuously extracting the native interstitial fluid through the skin of an animal or a human comprising means for a non-heating irreversible electroporation of the skin by applying a non-pulsed voltage coupled to a pulsed voltage between at least one moving electrode and a stationary electrode; a vacuum pump providing a negative pressure above the electroporated skin; and a collecting chamber for collecting the native interstitial fluid extracted through the skin.

In a further embodiment, the collecting chamber comprises an extraction chamber for collecting the native interstitial fluid extracted through the skin.

In another embodiment, the non-moving or stationary electrode serves at the same time as the positive electrode or the counter electrode and as an adhesive to attach the extraction chamber to the skin.

In another embodiment, the non-moving electrode serves at the same time as the positive electrode or the counter electrode, as an adhesive to attach the extraction chamber to the skin and as an adhesive to attach the extraction electronic interface to the skin.

In another embodiment, the non-moving electrode which serves at the same time as the positive electrode or the counter electrode and an adhesive to attach the extraction chamber to the skin and to attach the extraction electronic interface to the skin is made from a material such as a conductive double sided adhesive carbon tabs or tapes from for example Rave Scientific.

In another embodiment, the non-moving electrode which serves at the same time as the positive electrode or the counter electrode and an adhesive to attach the extraction chamber to the skin and to attach the extraction electronic interface to the skin is made from a material such as conductive double sided adhesive tabs or tapes where carbon material is substituted by a noble metal or any stable material at the applied positive voltage.

In another embodiment, the moving electrode which serves as the negative electrode or the electroporating or the active electrode is a noble metal or its alloy-based electrode.

In an embodiment, the method also comprises the step of analyzing the presence or the absence or the concentration of an analyte or a plurality of analytes in the extracted native interstitial fluid through the skin by a single frequency impedimetric sensor.

In a further embodiment, the analyte is Covid-19.

In a further embodiment, the negative pressure is comprised between 0.99 atm and 0.2 atm.

In an embodiment, the method also comprises the step of transmitting the analysis of the concentration of the analyte or its presence or its absence in the extracted native interstitial fluid through the skin by a wireless mean/device.

In another embodiment, the collection of the native interstitial fluid and the analysis of the concentration of the analyte in the native interstitial fluid extracted through the skin are regulated by a check valve, such as for example but not limited to, a one-way valve or the peristaltic pump when it is used.

In an additional embodiment, the negative pressure is pulsed or continuous.

Further, the negative pressure applied at or near the region of the non-heating irreversible electroporated skin can be monitored by a pressure sensor.

In another embodiment, the subject is an animal or a human.

In an embodiment, the device further comprises a biosensor or a plurality of sensors/biosensors for analyzing the concentration of an analyte or a plurality of analytes in the extracted native interstitial fluid through the electroporated skin.

The biosensor or the plurality of sensors can comprise an electrochemical sensor or an optical sensor; a wired or wireless communication device; a sensing electrode, a reference electrode and a counter electrode; and/or a sensing electrode and an accompanying electrode acting both as a reference and a counter electrode. Moreover, the device might include a flow sensor, a pressure sensor and a temperature sensor.

In a further embodiment, the biosensor or a plurality of sensors continuously analyze or detect the concentration or the presence or the absence of Covid-19 in the extracted interstitial fluid through the electroporated skin.

In an embodiment, the device further comprises a way to enhance the attachment of the device on the subject, such as a bracelet, a strap or a combination thereof.

In an embodiment, the device further comprises a pressure sensor for monitoring the pressure applied above the electroporated skin.

In another embodiment, the device further comprises a temperature sensor for monitoring the temperature of the extracted native interstitial fluid.

In another embodiment, the device further comprises a flow sensor for monitoring the flow of the extracted native interstitial fluid.

In an embodiment, a valve may or may not be used and disposed between the biosensor and the collecting chamber creating a discontinuity between the extracted interstitial fluid still in the extraction chamber and the interstitial fluid being analyzed by the biosensor.

In an embodiment, the pump is a peristaltic pump, a diaphragm pump, or a piston pump.

In another embodiment, the biosensor is obtained by deposing a mixture of anti-Spike or anti-envelope, bovine serum albumin or other serum albumin as a stabilizer and a blocker of undesirable biomaterials, a promotor of adhesion such as aminosilane and nano-particles of an electronic conductor such as Vulcan XC-72R from Cabot Corporation on the surface of a carbon electrode (sensing electrode).

In another embodiment, the biosensor has a reference electrode made of silver/silver chloride and a counter-electrode made of a noble metal or its alloy.

In another embodiment, the biosensor has a reference electrode made of silver/silver chloride which acts at the same time as a counter-electrode.

In an embodiment, the device further comprises a sampling chamber connected to the extraction chamber by a pumping conduit, the sampling chamber collects the extracted interstitial fluid from the skin for its analysis.

In another embodiment, the device further comprises a microcontroller connected to the pressure sensor in order to monitor and control the pressure above the skin and inside the extraction chamber.

In another embodiment, the non-heating irreversible electroporation of the skin is monitored by measuring the charge transfer impedance at the interface of a negative/active electrode and the interstitial fluid comparatively to a non-polarized electrode.

In another embodiment, all the components and devices are protected by different means such as a strong tape to make all the system waterproof.

In another embodiment, the non-heating irreversibly electroporated surface area may be comprised between 2 mm² to 500 cm².

In another embodiment, the flow rate of the extracted interstitial fluid may be comprised between 0.05 μL/minute to 5 mL/minute.

In another embodiment, the very low flow rate is used for the continuous monitoring of an analyte or a plurality of analytes in the extracted native interstitial fluid.

In another embodiment, the moderate to the high flow rate recited herein is used to produce antibodies, vaccine, biomarkers, dialysis, fractionation, etc. from and of the extracted native interstitial fluid.

### BRIEF DESCRIPTION OF THE DRAWINGS

Reference will now be made to the accompanying drawings, showing by way of illustration, a preferred embodiment thereof.

FIG. 1 illustrates an illustration of impedimetric sensor before and after the deposition of the sensing layer on the sensing electrode in accordance to an embodiment.

FIG. 2A illustrates a schematic representation of an electrical equivalent circuit between the sensing electrode and the reference electrode in accordance to an embodiment.

FIG. 2B illustrates a Nyquist plot obtained when an alternative voltage with a sinusoidal amplitude comprised between 2 to 60 mV is superimposed to the sensing electrode is maintained at a DC voltage corresponding to the rest voltage or the open circuit voltage of the electrochemical cell (impedimetric sensor) in agreement with the electrical equivalent circuit shown in FIG. 2A.

FIG. 3 illustrates a top view of the bottom of an extraction chamber as described herein in accordance to an embodiment.

FIG. 4 illustrates a planar top view of the bottom of the extraction chamber in accordance to an embodiment.

FIG. 5 illustrates a top view of the upper part of the extraction chamber in accordance to an embodiment.

FIG. 6 illustrates a top view of the upper part of the extraction chamber in accordance to an embodiment.

FIG. 7 illustrates an assembled view of one extraction chamber in conjunction with an electronic controller box as encompassed herein in accordance to an embodiment.

FIG. 8 illustrates an assembled view of one extraction chamber in conjunction with the electronic controller box in accordance to an embodiment.

FIG. 9 illustrates a top view of three extraction chambers connected in series to the same pump inside the electronic controller box in accordance to an embodiment.

It will be noted that throughout the appended drawings, like features are identified by like reference numerals.

### DETAILED DESCRIPTION

In accordance with the present invention, there is provided a device for non-invasively and continuously extracting interstitial fluid which may or may not comprise an immunogenic compound from the skin of a subject comprising an electroporator generating a non-pulsed voltage coupled to a pulsed voltage between at least one moving electrode and an electrical conducting adhesive for non-heating irreversibly electroporating the skin; a vacuum pump providing a negative pressure above the skin, the vacuum pump in operation providing the negative pressure simultaneously with electroporating the skin to form openings in the skin for extracting the interstitial fluid, the interstitial fluid being extractable only so long as the negative pressure is applied in combination with electroporating the skin; and a collecting chamber for collecting the interstitial fluid extracted from the skin.

The present disclosure describes a method for extracting the interstitial fluid from the people who recovered from the infection by viruses such as Covid-19, Ebola, etc. It is provided a method and devices for the early detection of Covid-19 in humans and animals before even the apparition of the first symptoms such as fever. The method described herein involves firstly production of anti-Spike or anti-envelope or Interleukin-6 (antibody) in animals such as mouse or rat, rabbit, hamster, guinea pig, goat, cow, horse, etc. by injecting the Spike protein/adjuvant conjugate or, the covid-19 envelope/adjuvant conjugate or Interleukin-6/adjuvant conjugate (antigen/adjuvant conjugate) or interleukin-6 (antigen/adjuvant conjugate) in the host animal. Afterwards the anti-Spike or the anti-envelope or the anti-interleukin-6 (antibody) from the interstitial fluid of the host animal are isolated. A continuous extractor of the interstitial fluid on the host animal is used in order to obtain the highest quantity of the anti-Spike or the anti-envelope or the anti-interleukin-6 from the host animal. An impedimetric sensor can be prepared by deposing a mixture of anti-Spike or anti-envelope or the anti-interleukin-6, bovine serum albumin or other serum albumin, a promotor of adhesion such as aminosilane and nano-particles of an electronic conductor such as Vulcan XC-72R on the surface of a carbon electrode (sensing electrode). Measurement of an electrical parameter generated when a sinusoidal potential perturbation is superimposed on a DC potential and applied to the impedimetric sensor maintained at a DC voltage which corresponds to the rest voltage or the open circuit voltage of the impedimetric sensor. Such electrical parameters might include the variation of the values of the impedance (from Nyquist plot) or the phase angle/phase shift (from Bode plot) at the interface sensing electrode/interstitial fluid.

The present disclosure relates to a modified method and devices for extracting continuously and non-invasively the interstitial fluid from an animal or a human. A method and device for extracting continuously and non-invasively the interstitial fluid from an animal or a human has been described previously in WO 2010/094131, the entire content of which is incorporated herein by reference. The method and apparatus described herein allows the reduction of the feeling of the electrical field on the skin to near zero, the elimination of the inflammation of the skin associated with its electroporation and the confinement of the micro-openings in order that they will be easy to heal after utilization without leaving any trace on the skin when compared to the early disclosure.

It is also provided the production of an antibody of an antigen from an animal. According to this application, the production of the antibody in the host animal takes place by extracting a sample of the interstitial fluid of the host animal, mixing the antigen/adjuvant conjugate to the extracted sample of the interstitial fluid containing the animal albumin which will act as a carrier for the antigen/adjuvant conjugate. Then the mixture is injected into the host animal by the mean of a transcutaneous injection. This procedure is repeated if it is needed until a vigorous immune response is obtained. Accordingly, the antibody is obtained by extracting non-invasively and continuously the interstitial fluid from the host animal and using the standard methods available in the market for its separation and purification from the extracted interstitial fluid.

The presents disclosure relates also to the fabrication of an impedimetric sensor which is coupled to a wearable device for the continuous and non-invasive extraction of the native interstitial fluid through the skin of an animal or a human. The measurement of an electrical parameter generated when a sinusoidal potential perturbation is superimposed on a DC potential and applied to the impedimetric sensor maintained at a DC voltage which corresponds to the rest voltage or the open circuit voltage of the impedimetric sensor. Such electrical parameters might include the variation of the values of the impedance/charge transfer impedance (from Nyquist plot) or the phase angle/phase shift (from Bode plot) at the interface sensing electrode/interstitial fluid.

More importantly, the methods and devices described in the present disclosure are intended to be attached to the people in the front line to deal with the Covid-19 such as the medical staff and volunteers, security personal, army and the people who travel and find themselves in very close contact with many people. An example of such people is those who travel by airplane, train, bus, ship cruiser, confined in hotels around the world, etc. In this application it is encompassed a methods for the production of an antibody of an antigen.

The antigen used in this disclosure, as an example, could be the Spike protein or S protein which is membrane glycoprotein (200-220 kDa) which occurs in the form of spicules or “spikes” emerging from the surface of the viral envelope. It is responsible for the attachment of the virus to receptors in the host cell and for the induction of fusion of the viral envelope with the cell membrane. In an embodiment, the envelope protein or E protein also called sM (small membrane) is used which is a non-glycosylated trans-membrane protein of approximately 10 kDa, wherein it is the protein present in the lowest amount in the virion. It plays a driving role in the budding process of coronaviruses which occurs at the level of the intermediate compartment in the endoplasmic reticulum and the Golgi apparatus. In an alternate embodiment, the interleukine-6 (IL-6) associated with the cytokine storm triggered by Covid-19 infection is used. The cytokine storm (hypercytokinemia) is the systemic expression of a healthy and vigorous immune system resulting in the release of more than 150 known inflammatory mediators. Cytokine storms have potential to do significant damage to body tissues and organs. If a cytokine storm occurs in the lungs, for example, fluids and immune cells such as macrophages may accumulate and eventually block off the airways, potentially resulting in death. Use of a continuous extractor of the interstitial fluid on the host animal in order to obtain the highest quantity of the anti-Spike or the anti-envelope or the anti-IL-6 from the host animal.

Isolation of the anti-Spike or the anti-envelope or the anti-IL-6 from the interstitial fluid of the host animal is also encompassed.

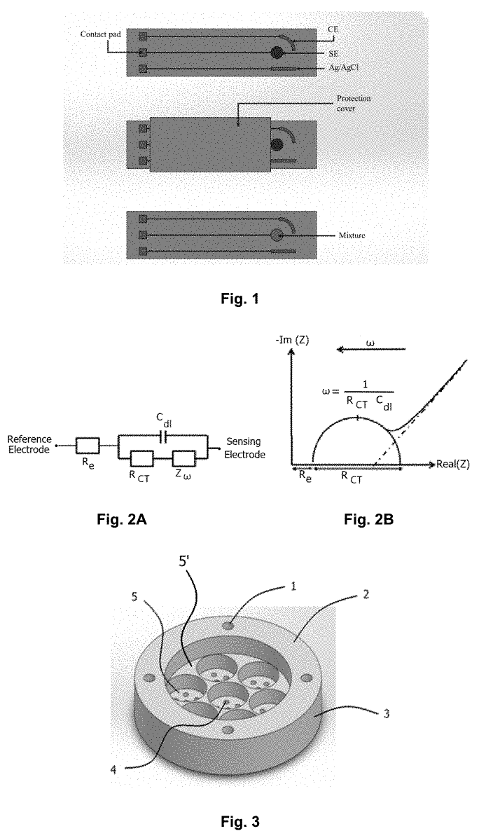

It is encompassed the preparation of an impedimetric sensor (see FIG. 1) by deposing a mixture of anti-Spike or anti-envelope, bovine serum albumin or other serum albumin as a stabilizer and a blocker of undesirable biomaterials, a promotor of adhesion such as aminosilane and nano-particles of an electronic conductor such as XC-72R on the surface of a carbon electrode (sensing electrode). The impedimetric sensor could be used with or without a counter-electrode.

It is described herein, measurement of an electrical parameter generated when a single frequency of an alternative voltage with a sinusoidal amplitude comprised between 2 to 60 mV is superimposed to the sensing electrode maintained at a DC voltage which corresponds to the rest voltage or the open circuit voltage of the electrochemical cell (impedimetric sensor). Such electrical parameters might include the variation of the values of the impedance (from Nyquist plot: FIGS. 2a and 2b) or the phase angle/phase shift (from Bode plot) at the interface sensing electrode/interstitial fluid when the presence the antigen (Covid-19) or its concentration varies at the interface sensing electrode/interstitial fluid.

The electrical elements as described herein (see FIG. 2A) comprise resistance of the electrolyte between the reference electrode and the sensing electrode (Rₑ) which is a pure ohmic resistance, a double layer capacitance (C_{dl}), a charge transfer resistance (R_{ct}) which is not an ohmic resistance and a Warburg diffusion element (Z_{ω}). Z_{ω} is a constant phase element (CPE), with a constant phase of 45° (phase independent of frequency) and with a magnitude inversely proportional to the square root of the frequency.

The Nyquist plot obtained when an alternative voltage with a sinusoidal amplitude comprised between 2 to 60 mV is superimposed to the sensing electrode is maintained at a DC voltage corresponding to the rest voltage or the open circuit voltage of the electrochemical cell (impedimetric sensor) in agreement with the electrical equivalent circuit shown in FIG. 2A is seen in FIG. 2B. This plot is obtained when the frequency, w, of the sinusoidal voltage has varied between 1 mHz to 100 KHz. The X axis is represented by the real values of the impedance (Real (Z)) and the Y axis is represented by the negative module of the imaginary values of the impedance (−Im (Z)) where Z is the impedance. In the plot in FIG. 2B, there is three distinct domains represented by an Ohmic resistance, Rₑ, a domain where the system is controlled by the charge transfer resistance, R_{ct}, at the interface extracted interstitial fluid and the sensing electrode with at least one time constant expressed by C_{dl}. In this disclosure, the value of the R_{ct} will increase with the increases of the concentration of the Covid-19 or its presence.

Currently, an antibody of interest is produced in a host animal by injecting an antigen/adjuvant conjugate into the host animal of choice to initiate an amplified immune response. This induces the B-lymphocytes to produce IgG immunoglobulins specific for the antigen. This polyclonal IgG is purified from the mammal's serum. After a series of injections over a specific length of time, the animal is expected to have created antibodies against the conjugate. Blood is then extracted from the animal and then purified to obtain the antibody of interest. Inoculation is performed on a suitable mammal, such as a mouse, rabbit, guinea pig, hamster, goat, etc. Larger mammals are often preferred as the amount of serum that can be collected is greater.

As known, the general procedure to produce polyclonal antibodies is as follows. Pre-immune serum from the mouse, as an example, is collected to use as a blank when performing ELISA screening after immunization. The antigen and adjuvant are prepared and animal selected. 50 to 100 μg of immunogen (equal to 100 to 200 μL of antigen-adjuvant mixture) are injected per mouse for example. Typical routes of injection include intraperitoneal (i.p.) or subcutaneous (s.c.). One or two such injections may be made per animal. The values cited here are for mouse. Therefore, more the animal is larger more the quantity of the antigen/adjuvant conjugate or immunogen in adjuvant is larger. Test bleed and assay antibody response are evaluated typically by ELISA (typically, mice are bled under anesthesia through the tail vein or the retro-orbital plexis). Boost with an equivalent amount of immunogen in adjuvant at different time intervals can be given if necessary and the procedure continues with a similar schedule of alternating boosts and test bleeds until a satisfactory response is observed. For monoclonal antibody production, either i.p. or intravenously (i.v.) injection are conducted 4 to 5 days before fusion the immunogen dissolves in saline solution (no adjuvant).

In an embodiment, the Spike protein or the envelope of the Covid-19 will be used as the antigen of the method described hereinabove. Furthermore, the anti-Spike, SARS-CoV-2 Spike Antibody, or the anti-envelope, SARS-CoV-2 Envelope Antibody, from ProSci Incorporated can be used.

Furthermore, it is encompassed the production of an impedimetric sensor for the continuous detection of the presence, absence or the concentration of a pathogen in the body of a human or an animal, such as for example but not limited to the Covid-19. The anti-Spike, SARS-CoV-2 Spike Antibody, or the anti-envelope, SARS-CoV-2 Envelope Antibody, from ProSci Incorporated can be used. SARS-CoV-2 Spike Antibody and SARS-CoV-2 Envelope Antibody are preferably diluted in a PBS solution in order to reach a 1 ng/mL concentration of the antibodies in the PBS solution.

In another embodiment, the biosensor is obtained by deposing a mixture of anti-Spike or anti-envelope (0.5 ng to 100 ng W/W), bovine serum albumin (1 μg to 1000 μg W/W) as a stabilizer and a blocker of undesirable biomaterials, a promotor of adhesion such as aminosilane (0.01 ng to 0.1 ng in 100 μL in ethanol solution) and nano-particles of an electronic conductor such as XC-72R on the surface of a carbon electrode (sensing electrode). The concentration of the XC-72R in the mixture must be comprised between 0.5 μg to 2 μg W/W.

In another embodiment, the biosensor is obtained by deposing the above mixture of anti-Spike or anti-envelope, bovine serum albumin or other serum albumin as a stabilizer and a blocker of undesirable biomaterials, a promotor of adhesion such as aminosilane and nano-particles of an electronic conductor such as XC-72R on the surface of a noble metal or its alloy based electrode (sensing electrode).

In another embodiment, the biosensor has a reference electrode made of silver/silver chloride and a counter-electrode made of a noble metal or its alloy.

In another embodiment, the biosensor has a reference electrode made of silver/silver chloride which acts at the same time as a counter-electrode.

Disclosed herein is a method which creates irreversibly as many electrophilic conduits through the epidermis as needed. These openings are permanent, irreversible and have a duration limited only by the duration of the extraction of the native interstitial fluid. Otherwise, these openings or conduits through the epidermis are the subject of a controlled vacuum. Disclosed herein is a method which creates one or more electrophilic conduit through the epidermis. These openings are permanent and have a duration limited only by the duration of the treatment.

Contrary to the device and a method described in Canadian patent No. 2655017, incorporated by reference herewith in its entirety, the device provided herein comprises major modifications to irreversibly form one or a plurality of openings or conduits solely through the epidermis. These major modifications relate to the fact that a solid positive electrode is no more used, and this solid positive electrode is replaced by an electrical conducting adhesive and could be placed anywhere around or near the extraction chamber. The conduit(s) hence formed will form the means by where the native interstitial fluid is extracted for further processing.

As encompassed herein, the term “irreversibly” is intended to describe real conduits formed through the epidermis. These conduits last if a vacuum is maintained above the part of the skin where the openings are located. These conduits have real physical dimensions such as a diameter and a depth. They are irreversible.

The openings or conduits formed when the method described herein is applied is intended to mean micro-holes through the epidermis formed following a short and controlled non-heating irreversible electroporation as described herein. Non-heating irreversible electroporation is the process by which and when applied on the skin of a living subject lead to the irreversible formation of micro-holes through the epidermis. The formed micro-holes last if they are needed.

More particularly, the device encompassed herein comprises means for electroporating the skin by generating a non-pulsed voltage coupled to a pulsed voltage at a small current between at least two electrodes where one of them is not a solid electrode and which is the positive electrode. The non-heating irreversibly electroporation described herein of a region of the skin is done without generating any heat to preserve the connective tissue, in order to not denature molecules and collagen, eliminating any injuries to the cell scaffold and does not compromise the blood vessel matrix, which results in a clear margination of treated and non-treated areas.

Electroporation of the skin is affected by the amplitude of the applied voltage, the shape of the applied voltage and of the electrodes, the frequency of the applied signal, the intensity of the applied current and the duration of the applied signal. The amplitude of the applied voltage is expressed in volts and can vary from 5 V to 500 V depending on the above variables and it is now not independent on the distance between the positive and the negative electrodes if this distance is comprised between 1 to 10 cm. The frequency of the applied voltage may vary from 50 μS to 7000 μS. The shape of the signal is preferred to be square but other types of signal can be used. The preferred duration is less than three minutes depending on the above cited variables, but duration above the preferred value or under the preferred value is not excluded.

Herein, non-heating irreversible electroporation is an event in which microsecond electrical pulses are applied on a living or non-living tissue, wherein the tissue is illustrated by the epidemies of a living subject, destabilizing the electrical potential across the cell membrane and resulting in irreversible nanoscale/microscale pores. Accordingly, to avoid the non-desirable Joule's effect or the generation of heat, the electrical parameters are chosen such that the cell membrane is selectively targeted without inducing thermal damage to the rest of the tissue. These parameters are illustrated hereinafter in Table 1.

TABLE 1
Example of the Preferred Electrical Parameters for two electrodes

| Parameters | Unit values |
|---|---|
| Applied pulsed voltage | 50 to 200 V |
| Applied DC voltage | 0.5 to 20 V |
| Distance between two negative electrodes | 1 to 100 mm |
| Frequency | 50 to 7000 μS |
| Current | 5 to 70000 μA |
| Duty cycle/pulse | 0.05 to 27% |
| Duration | 60 to 1700 Seconds |
| Time between each 60 s of electroporation | 15 to 20 s |
| Measurement of the resistance | after each 60 Seconds |
| | Between negative electrodes |

Due to its non-heating nature, the irreversible electroporation described herein does not affect the connective tissue nor does it denature molecules and collagen, eliminating any injuries to the cell scaffold and does not compromise the blood vessel matrix, and that the irreversible electroporation results in a clear margination of treated and non-treated areas.

The present disclosure describes a method to monitor the time when the non-heating irreversible electroporation has opened the micro-conduits through the epidermis and at this time the process of the electroporation must end. The monitoring of the end of the process of the electroporation may be continuous by measuring the increase in the applied voltage, the value of the pulsed voltage plus the value of the DC voltage, or by measuring the increase of the applied current or by measuring the resistance between two negative electrodes after each 60 seconds of electroporation. The measurement of the resistance between two adjacent negative electrodes is carried out when the process of the electroporation is stopped at each 60 seconds interval. The measurement of the resistance between two adjacent negative electrodes is done by using a high input impedance ohmmeter, 1 TΩ, integrated in the electronic interface which control all the process of the electroporation. The measurement of the resistance could be done by using the ohmmeter or the value of the resistance obtained when a sinusoidal potential perturbation is superimposed on a DC potential and applied to the two adjacent electrodes maintained at a DC voltage which corresponds to the rest voltage or the open circuit voltage between the two electrodes. The preferred value of the amplitude of the sinusoidal perturbation is 10 mV±2 mV at a frequency comprised between 100 Hz to 100 KHz.

The present disclosure describes a method for the noninvasive and continuous extraction of the interstitial fluid through the skin. This method as will be seen hereinafter provides an accurate way to obtain openings that last for days or if a vacuum is applied above the electroporated area. The openings according to the present disclosure are in the order of micrometers, which is different than what is described in the actual art in the order of nanometers. Moreover, the effectiveness of the method described in the present description has been verified by applying it on five human volunteers with one of them being a healthy person, another diabetic of type 2, another performs routinely hemodialysis and three different animals (rabbit, donkey and a cow). The analysis of some analytes in the extracted native interstitial fluid is shown in the Tables 2 to 5 hereinafter. The electroporation as described in the present disclosure is a non-heating irreversible electroporation which lead to irreversible openings of the skin or more specifically the epidermis. The openings last for days, hence, an accurate and precise monitoring of one or a plurality of analytes in the extracted interstitial fluid can be carried out with and without further electroporation of the same area of the skin (Tables 2 to 4). The present disclosure provides the application of a non-pulsed DC voltage as explained hereinbelow between the electrodes at a fixed current at the same time of the applications of the pulsed voltage necessary for the irreversible electroporation and between the pulses.

The present disclosure describes a method and devices for the non-heating irreversible electroporation of a surface area of the epidermis comprised between 2 mm² to 500 cm². Therefore, since the flow rate of the extracted native interstitial fluid depends mostly on the electroporated surface area of the epidermis or the number of the micro-openings, the flow rate of the extracted native interstitial fluid may be comprised between 0.05 μL/minute to 5 mL/minute. Herein, the very low flow rate is used for the continuous monitoring of an analyte or a plurality of analytes in the extracted native interstitial fluid or other use which does not require a moderate to high flow rate of the extracted native interstitial fluid. In the present disclosure, the moderate to the high flow rate cited hereinabove is used to produce antibodies, vaccine, biomarkers, dialysis, fractionation, etc. from and of the extracted native interstitial fluid.

TABLE 2
Value of some biomarkers of a healthy person
Biomarkers in subject's interstitial fluid
General Biochemistry

| | | |
|---|---|---|
| Urea | 7.5 | mM/L |
| Creatinine | 73 | μM/L |
| Sodium | 161 | mM/L |
| Potassium | 4.9 | mM/L |
| Chlorides | 126 | mM/L |
| Total CO₂ | 26 | mM/L |
| Total calcium | 1.38 | mM/L |
| Ionized calcium | 0.67 | mM/L |
| Total Bilirubin | 5 | μM/L |
| Total Proteins | 35 | g/L |
| Albumin | 25 | g/L |
| AST | 48 | U/L |
| ALT | 9 | U/L |
| GGT | 5 | U/L |
| Alkaline Phosphatase | 18 | U/L |
| Lactic acid | 5.2 | mM/L |
| C-reactive protein | <0.2 | mg/L |

Erythropoiesis

| | | |
|---|---|---|
| Vitamin B12 | 341 | pM/L |
| Folic acid | >45.4 | nM/L |

Specific proteins: Immunoglobulins

| | | |
|---|---|---|
| IgG | 3.12 | g/L |
| IgA | 0.40 | g/L |
| IgM | 0.31 | g/L |

Endocrinology

| | | |
|---|---|---|
| Cortisol | 115 | nM/L |

Biomarkers in subject's blood
General Biochemistry

| | | |
|---|---|---|
| Urea | 4.3 | mM/L |
| Creatinine | 93 | μM/L |
| Sodium | 138 | mM/L |
| Potassium | 3.8 | mM/L |
| Chlorides | 102 | mM/L |
| Total CO₂ | 26 | mM/L |
| Total calcium | 2.52 | mM/L |
| Ionized calcium | 1.22 | mM/L |
| Total Bilirubin | 12 | μM/L |
| Total Proteins | 73 | g/L |
| Albumin | 48 | g/L |
| AST | 28 | U/L |
| ALT | 19 | U/L |
| GGT | 11 | U/L |
| Alkaline Phosphatase | 37 | U/L |
| Lactic acid | 2.6 | mM/L |
| C-reactive protein | <0.2 | mg/L |

Erythropoiesis

| | | |
|---|---|---|
| Vitamin B12 | 310 | pM/L |
| Folic acid | 25.4 | nM/L |

Specific proteins: Immunoglobulins

| | | |
|---|---|---|
| IgG | 7.45 | g/L |
| IgA | 1.17 | g/L |
| IgM | 1.17 | g/L |

Endocrinology

| | | |
|---|---|---|
| Cortisol | 565 | nM/L |

TABLE 3
The values of some biomarkers present in the interstitial fluid and the
blood of a volunteer before and after the hemodialysis of his blood.

| | Interstitial | Interstitial | | |
|---|---|---|---|---|
| | fluid before the | fluid after the | Blood before the | Blood after the |
| Biomarker | hemodialysis | hemodialysis | hemodialysis | hemodialysis |
| Alkaline reserves (Bicarbonate) mM/L | 19.5 | 20.2 | 22.3 | 29.4 |
| Urea mM/L | 28.8 | 20.1 | 22.1 | 14.1 |
| Creatinine mg/L | 110.11 | 64.79 | 101.82 | 54.34 |
| CPK IU/L | 144.0 | 74.0 | 123.0 | 116.0 |
| Glucose g/L | 1.28 | 1.29 | 0.93 | 1.42 |
| LDH IU/L | 243 | 243 | 198 | 179 |
| Sodium mM/L Potassium mM/L | 122.8 | 124.4 | 128.0 | 137.6 |
| Chloride ion mM/L | 5.90 | 5.53 | 5.48 | 4.59 |
| | 88.0 | 92.8 | 84.4 | 88.9 |
| Transaminases (ASAT) IU/L | 19.4 | 16.4 | 15.3 | 14.9 |
| Transaminases (ALAT) IU/L | 6.6 | 5.1 | 7.7 | 8.2 |
| Total Cholesterol Total g/L Cholesterol | 0.38 | 0.21 | 1.58 | 1.4 |
| HDL g/L Cholesterol LDL g/L | 0.10 | 0.10 | 0.30 | 0.30 |
| | 0.21 | 0.06 | 1.02 | 0.86 |
| Triglycerides g/L | 0.33 | 0.24 | 1.30 | 1.34 |
| PSA ng/mL | 0.356 | 0.327 | 0.815 | 0.784 |
| Albumin g/L | 18.10 | 12.10 | 47.00 | 43.00 |
| Gamma-GT IU/L | 8.0 | 5.0 | 33.0 | 39.0 |
| C Reactive Protein mg/L | 0.01 | 0.01 | 0.89 | 0.95 |

TABLE 4
The values of some biomarkers present in the interstitial fluid and the
blood of a volunteer before and after the hemodialysis of his blood.

| | Interstitial | Interstitial | | |
|---|---|---|---|---|
| | fluid before the | fluid after the | Blood before the | Blood after the |
| Biomarker | hemodialysis | hemodialysis | hemodialysis | hemodialysis |
| Alkaline reserves (Bicarbonate) mM/L | 19.5 | 20.2 | 22.3 | 29.4 |
| Urea mM/L | 28.8 | 20.1 | 22.1 | 14.1 |
| Creatinine mg/L | 110.11 | 64.79 | 101.82 | 54.34 |
| CPK IU/L | 144.0 | 74.0 | 123.0 | 116.0 |
| Glucose g/L | 1.28 | 1.29 | 0.93 | 1.42 |
| LDH IU/L | 243 | 243 | 198 | 179 |
| Sodium mM/L Potassium mM/L | 122.8 | 124.4 | 128.0 | 137.6 |
| Chloride ion mM/L | 5.90 | 5.53 | 5.48 | 4.59 |
| | 88.0 | 92.8 | 84.4 | 88.9 |
| Transaminases (ASAT) IU/L | 19.4 | 16.4 | 15.3 | 14.9 |
| Transaminases (ALAT) IU/L | 6.6 | 5.1 | 7.7 | 8.2 |
| Total Cholesterol Total g/L | 0.38 | 0.21 | 1.58 | 1.4 |
| Cholesterol HDL g/L Cholesterol | 0.10 | 0.10 | 0.30 | 0.30 |
| LDL g/L | 0.21 | 0.06 | 1.02 | 0.86 |
| Triglycerides g/L | 0.33 | 0.24 | 1.30 | 1.34 |
| PSA ng/mL | 0.356 | 0.327 | 0.815 | 0.784 |
| Albumin g/L | 18.10 | 12.10 | 47.00 | 43.00 |
| Gamma-GT IU/L | 8.0 | 5.0 | 33.0 | 39.0 |
| C Reactive Protein mg/L | 0.01 | 0.01 | 0.89 | 0.95 |

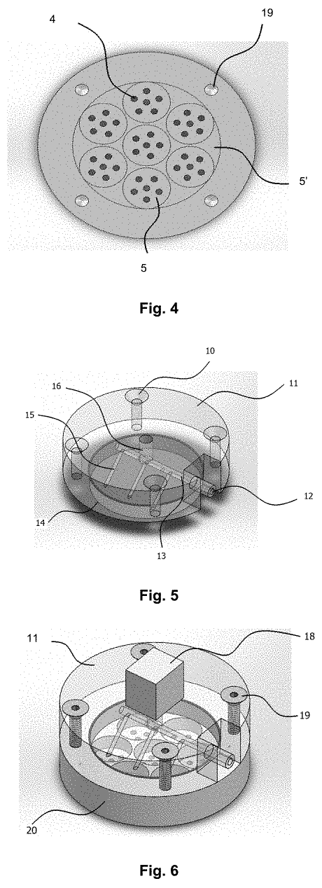

An embodiment of the extraction chamber of the device encompassed herein is illustrated in FIGS. 3 to 9, showing a typical application of the method and is designed to be used for occasional or continuous sampling of the extracted transdermal fluid or simply its continuous extraction for other purposes. The sampling method could be manual or automated. The device comprises an insulating body (see FIGS. 3; 5, 5′, 2 and 3) maintained in contact with the skin through the epidermis using an adhesive or any other suitable sealing product or maintaining method 26.

The extraction chamber comprises at least one hole 1 to receive a screw 19, a body 3 of the extraction chamber is made of a polymer body 2 with holes 4, wherein negative solid electrodes passes through said holes 4 to be in contact with the skin. A thin layer membrane 5 of 50 to 200 μm is used to keep the skin in place and to avoid its suction. A further thicker membrane 5′ of 2 to 5 mm is used to separate the different extraction mini-chamber.

As seen in FIG. 5, in the upper part of the extraction chamber, a polymer is used to make the upper part 11 of the extraction chamber comprising the receiving ends 10 of holes that receive screws 19. An outlet of the extraction chamber 12 is connected to the inlet of the pump. A collection micro-conduit 13 serves to collect the extracted native interstitial fluid from the micro-conduits 15 and serves, also, to accommodate the pressure sensor 16. The connection between the bottom and the upper part of the extraction chamber is made by the platform 14.

The extraction chamber 21 can be made completely from a polymeric material, a combination of a polymeric material or any other material that are electrically non-conductive or insulating.

As seen in FIG. 6, a cape 18 is used to protect a pressure sensor, wherein screws 19 to fix the bottom to the upper part 20 of the extraction chamber.

The device can be covered by a material such as a capsule or an adhesive in order to facilitate its use in a swimming pool, during the use of a bath or a shower and/or any situation where a liquid can surround the extraction chamber 21. The extraction chamber 21 can include a sealed hole so that the collected freshly extracted fluid can be analyzed remotely from the device. Furthermore, in order to increase the fixture of the extraction chamber 21 as described herein, a non-allergenic material such as a bracelet or a strap can be used to stabilize the contact between the device and the skin.

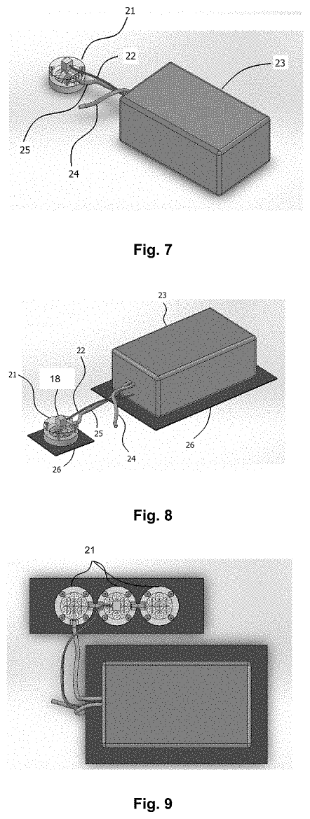

FIG. 7 illustrates in accordance to an embodiment an assembled view of one extraction chamber 21 in conjunction with an electronic controller box 23. The extraction chamber 21, is connected to the electronic controller box 23 containing the pump enclosed therein by an electrical cable 22 connecting the pressure sensor to the electronic controller box 23, wherein an outlet 24 and inlet 25 of the pump enters said electronic controller box 23.

Before the application of the extraction chamber 21 on the skin, the skin can be gently cleaned by any known chemicals used in medicine to clean such skin or simply using a soap and/or water. Preferably, any chemicals that evaporate after cleaning are used such that they do not leave any residue at the surface of the skin. Moreover, the chemicals or the soap should not induce any allergenic reaction of the skin nor modify the structure of the skin. Furthermore, in some cases the use of Povidone-iodine (PVP-I), also known as odopovidone must be used for kin disinfection before the electroporation and the installation the extraction chamber 21 and after the end of the extraction of the native interstitial fluid. As soon as the skin is gently cleaned or preferably disinfected, the extraction chamber 21 is attached on the top of the cleaned or disinfected skin by the help of the non-allergenic material such as the adhesive 26 or the strap or bracelet (FIG. 7). The adhesive 26 may or may not be based on an electrically conductive adhesive.

The preferred part of the skin is any part of the skin covering the arm of a person, but most of the body skin can be used with the device and the method described herein.

In an embodiment, pumping conduits can be in contact with a least one mini-sampling chamber and the complete extraction chamber 21, where the chosen sampling device shall be able to collect samples of the extracted transdermal fluid. The collection can be carried out, for example through a micros conduit which allows for continued extraction of the native interstitial fluid of a sealed sampling chamber 21. The sampling chambers, pumping conduits and extraction chamber 21 may or may not be isolated from a pump for example by a check valve or a one-way valve, thus creating a discontinuity between the analyzed fluid and the freshly extracted fluid which is to be analyzed. The check valve can be any means that creates or help to achieve a discontinuity between the analyzed fluid and the freshly extracted fluid such check valve could be the pump itself when it is a peristaltic pump. The extraction is continuously carried out by the application of a controlled negative pressure that is applied in the conduit system comprised namely of the extraction chamber 21, the extraction or the pumping conduit and the sampling chambers. The controlled negative pressure is maintained by the action of the pump and the level of the negative pressure is continuously monitored and controlled using a pressure sensor or pressure switch, which could be located before or after the check valve.

The negative pressure is attained by activating the pump in an on/off fashion or in a continuously modulated fashion. The actual value of the negative pressure can be controlled using the pressure sensor or a pressure switch. The pump and the pressure sensor can be under the control of a microcontroller that shall continuously or discontinuously monitor the actual pressure in the device from reading the actual status of the pressure sensor or the pressure switch and shall accordingly activate or deactivate the operation of the pump in a continuous, discontinuous or modulated fashion in order to achieve the desired negative pressure. A temperature sensor can also be incorporated in order to improve the efficiency of the device and to correct the reading of the biosensor or the plurality of biosensors if needed. Furthermore, a flow sensor could be installed between the outlet of the extraction chamber 21 and the pump in order to inform the user that the process of the extraction is going well.

The vacuum is generated inside the chamber 21 between the skin and the pump in order to improve the adhesion, the continuous extraction of the transdermal fluid, the circulation of the extracted fluid and, consequently, the continuous monitoring of one analyte or a plurality of analytes in the extracted native interstitial fluid or simply the collection of the native interstitial fluid. Preferably, the vacuum pump can provide a vacuum that will provide enough suction to stretch the portion of the skin in the region from which the sample of interstitial fluid is to be extracted. As the suction provided by the vacuum pump is stretching the appropriate portion of the skin, the suction provided by the vacuum pump also causes the stretched portion to become filled with interstitial fluid. A vacuum pump that is suitable for the device defined herein can be a peristaltic pump, a diaphragm pump, a piston pump, a rotary vane pump, or any other pump that will perform the required functions set forth previously. Typically, the vacuum pump preferably employs a self-contained permanent magnet DC motor. Vacuum pumps that are suitable for this invention are well-known to those of ordinary skill in the art and are commercially available. The vacuum pump is preferably capable of providing a differential pressure down to about 0.90 atm, and is more preferably operated at from about 0.99 atm and 0.2 atm. The vacuum provided by the vacuum pump can be continuous or pulsed. It is preferred that the applied vacuum does not cause irreversible damage to the skin. It is preferred that the applied vacuum does not produce bruises and discolorations of the skin that persist for several days. It is also preferred that the level of applied vacuum and the duration of application of the vacuum do not be so excessive that it causes the dermis to separate from the epidermis, which results in the formation of a blister filled with fluid.

The irreversible electroporation of the skin is carried out by the application of a pulsed voltage between two electrodes or a plurality of electrodes forming a network of electrodes. As an illustration of the art herein, the excitation part of the process is carried out using at least two electrodes placed in contact with the exposed portion of the skin which is the negative electrode and the positive electrode which is used also as an adhesive. One of the excitation electrodes (negative electrode or electroporating electrode) is maintained in contact with the exposed portion of the skin during the non-heating irreversible electroporation.

The target result of the irreversible electroporation herein is an effective and non-invasive electropermeabilization of the epidermis leading to permanent micro-opening(s) as long as the suction is applied, without any pain felling and which can permit a continuous transdermal fluid extraction for as long as needed. Herein, the transdermal fluid is the native interstitial fluid. A continuous fluid extraction is intended herein to refer to a continuous flow of the interstitial liquid through the skin, including the opening(s) in the epidermis, into the extraction chamber 21.

As described herein, the irreversible electroporation is done by varying and actuating different parameters which include: the amplitude of a non-pulsed DC voltage, the intensity of the current flowing between the electrodes through the skin (l/mA), the amplitude of the pulsed applied voltage, the period of the pulses (μs), the duty cycle (%) and the duration time (s). The non-pulsed DC voltage can be defined as the minimum value of the pulsed voltage before and between the pulses. This voltage can be applied at the same current intensity a few seconds before the pulses or immediately at the starting of the non-heating irreversible electroporation. Table 1 shows the typical values associated with all the above different parameters.

Further, the present disclosure describes the production of an antibody of interest in a host animal by injecting an antigen/adjuvant conjugate mixed with the extracted native interstitial fluid of the same animal into the host animal of choice to initiate a vigorous immune response. After a series of injections over a specific length of time, the animal is expected to have created antibodies against the antigen. Native interstitial fluid is then extracted from the host animal and the antibody is purified by using the available methods available in the market. The inoculation is performed on a suitable mammal, such as a mouse, rabbit, guinea pig, hamster, goat, etc. Larger mammals may be used if the amount of native interstitial fluid that can be collected is greater.

While the disclosure has been described with particular reference to the illustrated embodiment, it will be understood that numerous modifications thereto will appear to those skilled in the art. Accordingly, the above description and accompanying drawings should be taken as illustrative and not in a limiting sense.

While the disclosure has been described in connection with specific embodiments thereof, it will be understood that it is capable of further modifications and this application is intended to cover any variations, uses, or adaptations including such departures from the present disclosure as come within known or customary practice within the art and as may be applied to the essential features hereinbefore set forth, and as follows in the scope of the appended claims.

What is claimed is:

1. A device for non-invasively and continuously extracting interstitial fluid comprising or not comprising an immunogenic compound from the skin of a subject comprising:
an electroporator generating a non-pulsed voltage coupled to a pulsed voltage between at least one moving electrode and an electrical conducting adhesive for non-heating irreversibly electroporating the skin;
a vacuum pump providing a negative pressure above the skin, the vacuum pump in operation providing the negative pressure simultaneously with electroporating the skin to form openings in the skin for extracting the interstitial fluid, the interstitial fluid being extractable only so long as the negative pressure is applied; and
a collecting chamber for collecting the interstitial fluid extracted from the skin.

2. The device of claim 1, comprising an impedimetric sensor for detecting the immunogenic compound.

3. The device of claim 2, wherein the impedimetric sensor comprises at least one of an anti-Spike antibody, anti-envelope antibody, anti-interleukin-6 antibody, a serum albumin and a promotor of adhesion.

4. The device of claim 3, wherein the promotor of adhesion is aminosilane or nano-particles of an electronic conductor.

5. The device of claim 1, the collecting chamber comprises an extraction chamber for collecting the native interstitial fluid extracted through the skin.

6. The device of claim 1, further comprising a sampling chamber connected to the extraction chamber by a pumping conduit, the sampling chamber collects the extracted interstitial fluid from the skin for its analysis.

7. The device of claim 1, further comprising a pressure sensor for monitoring the pressure applied above the electroporated skin.

8. The device of claim 7, further comprising a microcontroller connected to the pressure sensor in order to monitor and control the pressure above the skin and inside the extraction chamber.

9. The device of claim 1, said device non-heating irreversibly electroporating a surface area of the skin comprised between 2 mm² to 500 cm².

10. The device of claim 1, extracting interstitial fluid at a flow rate between 0.05 μL/minute to 5 mL/minute.

11. The device of claim 2, wherein the impedimetric sensor detects the immunogenic compound by using a micro-potentiostat which apply a single frequency sinusoidal potential perturbation superimposed on a DC potential and applied to the impedimetric sensor maintained at a DC voltage.

12. The device of claim 11, wherein the amplitude of the sinusoidal perturbation is 10 mV±2 mV and the single frequency is comprised between 10 mHz to 10 Hz

13. The device of claim 2, wherein the impedimetric sensor detects the presence of the Covid-19 virus.

14. A method of extracting an immunogenic compound from the skin from a subject comprising the steps of:
contacting the device of claim 1 on the skin of the subject;
electroporating the skin non-invasively and continuously extracting interstitial fluid from a subject;
applying a negative pressure at or near the region of the skin being electroporated, the simultaneous combination of electroporating the skin and applying the negative pressure forming openings in the skin or for suturing the skin and for extracting the interstitial fluid, the interstitial fluid being extractable only so long as the negative pressure is applied; and
extracting an immunogenic compound present in the interstitial fluid.

15. The method of claim 14, wherein the immunogenic compound is an antibody, an antigen, or a viral particle.

16. The method of claim 14, wherein the viral particle is from Covid-19 virus or Ebola virus.

17. The method of claim 14, wherein the antibody is an anti-Spike antibody, an anti-envelope antibody or Interleukin-6 antibody.

18. The method of claim 17, wherein the skin is electroporated by applying a voltage comprised between 5 V to 500 V.

19. The method of claim 18, wherein the frequency of the voltage is between 50 μS to 7000 μS.

20. The method of claim 14, wherein the subject is an animal or a human.
